(19) United States
(12) Patent Application Publication
(10) Pub. No.: US 2025/0264469 A1
(43) Pub. Date: Aug. 21, 2025

(54) METHODS FOR IMPROVING OUTCOMES FOR HEMATOPOIETIC CELL TRANSPLANT RECIPIENTS AT RISK FOR BRONCHIOLITIS OBLITERANS SYNDROME

(71) Applicant: CHILDREN'S HOSPITAL MEDICAL CENTER, Cincinnati, OH (US)

(72) Inventors: ASSEM ZIADY, Newport, KY (US); KASIANI MYERS, Cincinnati, OH (US); MICHELE WANG, Cincinnati, OH (US); MATTHEW SIEFERT, Cincinnati, OH (US); STELLA M. DAVIES, Cincinnati, OH (US)

(21) Appl. No.: 18/859,287

(22) PCT Filed: Apr. 28, 2023
(86) PCT No.: PCT/US23/20341

§ 371 Date: Oct. 23, 2024

Related U.S. Application Data
(60) Provisional application No. 63/336,036, filed on Apr. 28, 2022.

(51) Int. Cl. G01N 33/573; G01N 33/68

(52) U.S. Cl. CPC G01N 33/573; G01N 33/6893; G01N 2333/912; G01N 2800/122; G01N 2800/245; G01N 2800/50

(57) ABSTRACT

Disclosed are methods for treating an individual at risk for developing bronchiolitis obliterans syndrome (BOS) after hematopoietic stem cell transplant (HSCT), comprising a) detecting a biomarker; b) quantifying a biomarker level; and c) comparing the level of a biomarker to a control value; wherein a deviation in a level of biomarker indicates that said individual is likely to develop the lung condition.

### CROSS-REFERENCE TO RELATED APPLICATIONS

This application claims priority to and benefit of U.S. Provisional Application Ser. No. 63/336,036, filed Apr. 28, 2022, the contents of which are incorporated in their entirety for all purposes.

### STATEMENT REGARDING FEDERALLY-SPONSORED RESEARCH

This invention was made with government support under HL153108 awarded by the National Institutes of Health. The government has certain rights in the invention.

### BACKGROUND

Hematopoietic stem cell transplantation (HSCT) is a potentially curative treatment for many hematologic malignancies and nonmalignant disorders, including bone marrow failure syndromes, primary immunodeficiencies, and metabolic disorders. HSCT involves the infusion of hematopoietic stem cells from a donor to a recipient, with the goal of restoring normal hematopoietic function in the recipient. Despite significant advances in the field, HSCT remains associated with a high risk of morbidity and mortality due to a variety of complications. One such complication is bronchiolitis obliterans syndrome (BOS), a serious and potentially life-threatening pulmonary disorder that affects a significant proportion of HSCT recipients. BOS is characterized by inflammation and fibrosis of the small airways in the lungs, leading to airway obstruction and a progressive decline in lung function. While the exact pathogenesis of BOS remains unclear, it is thought to result from a complex interplay between the donor immune system and the recipient's lung tissue.

BOS occurs in approximately 10% of children after hematopoietic stem cell transplant (HSCT) and is commonly, although not always associated with graft versus host disease (GvHD). GvHD is a frequent complication after HSCT, and most children with GvHD will not develop BOS. Currently the factors that determine whether BOS does or does not occur in GvHD are unknown. Mortality rates from BOS in pediatric HSCT patients are reported as high as 22% while the morbidity is significant, in large part due to a delay in diagnosis.

The diagnosis of BOS in HSCT recipients can be challenging, as symptoms may be nonspecific and may overlap with other respiratory conditions. However, early recognition and prompt intervention are critical in managing BOS and improving outcomes. Currently, spirometry is the strategy of choice for diagnosing post-HSCT pulmonary complications. Several studies in adult HSCT recipients reported that spirometric changes, including FEF25-75% and FEV1 in the first 3 to 6 months after HSCT predicts late onset non-infectious pulmonary complications (LONIPC). The GvHD scoring system for detecting BOS incorporates alterations in FEV1, FEV1/FVC and FEF25-75% after HSCT (8). Spirometry presents major challenges in children due to the inconsistent effort and coordination performing a forced expiratory maneuver. Although spirometry may be accurate in children older than age 6 after considerable training, results can often be unsatisfactory. Pediatric HSCT recipients are often acutely ill and even older children are unable to perform reliable spirometry. Although spirometry is used successfully in other pediatric populations, such as cystic fibrosis, those particular patients are introduced to pulmonary function testing earlier in childhood and perform testing regularly, whereas pediatric HSCT recipients have inferior success rates due to sporadic use, even in teenagers. Lack of reliable spirometry in most children after HSCT often delays the identification of pulmonary complications, especially BOS, resulting in irreversible lung injury before therapeutic intervention can be implemented.

Currently, it is not possible to determine which children with GvHD will or will not go on to develop BOS. More importantly, the mechanism of BOS initiation is poorly understood and difficult to study as the disease is not commonly diagnosed until well established and end-organ damage has already occurred. Given the limitations of existing diagnostic modalities of BOS and GvHD in children after HSCT, novel approaches are needed to facilitate early, accurate diagnosis of BOS and timely treatment. Biomarkers for lung graft-versus-host-disease associated bronchiolitis obliterans syndrome in children following hematopoietic stem-cell transplant do not currently exist, and improved treatment for individuals having, or at risk of having BOS or BvHD are needed. Thus, there is a need for the identification of diagnostic biomarkers and treatments to improve the diagnosis and management of such conditions in HSCT recipients. The instant disclosure seeks to address one or more of the aforementioned needs in the art.

### BRIEF SUMMARY

Disclosed are methods for treating an individual at risk for developing bronchiolitis obliterans syndrome (BOS) after hematopoietic stem cell transplant (HSCT), comprising a) detecting a biomarker; b) quantifying a biomarker level; and c) comparing the level of a biomarker to a control value; wherein a deviation in a level of biomarker indicates that said individual is likely to develop the lung condition.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIGS. 1-3 depict a network analysis of markers that segregate (A) GvHD with BOS vs non-GvHD, (B) GvHD with BOS vs GvHD, and (C) GvHD vs non-GvHD prior to clinical diagnosis. Non-biased randomized and blinded proteomic analyses of banked plasma samples collected ˜68 days before clinical diagnosis of BOS from 3 cohorts of HSCT patients that went on to develop no GvHD, GvHD, or GvHD with BOS. MS label free quantitation was used to quantitate plasma protein isoform expression and the candidates that segregated patients that went on to develop GvHD or GvHD with BOS. Pathway analysis was performed on proteins that differed (p<0.05) or were present in one cohort and absent in the others. A number of these changes clustered around 9 markers: integrin-linked protein kinase (ILK), Kelch-like protein 5 (KLHLS), kinesin-like protein 22 (KID), SAC3 domain-containing protein 1 (SAC3D1), RRP12-like protein (PRP12), manganese transporting protein ATPase13A1 (ATP13A1), sorting nexin 8 (SNX8), and caspase 8 associated protein 2 (FLASH). Created with BioRender.com.

FIG. 4 depicts Go Process analysis of differential protein expression (p-value <0.10) in GvHD with BOS and non-GvHD (cohort B vs cohort A). Non-biased randomized and blinded proteomic analyses of banked plasma samples collected ˜68 days before clinical diagnosis of BOS compared to controls without GvHD. MS label free quantitation was used to quantitate plasma protein isoform expression and the candidates that segregated patients that went on to develop GvHD with BOS vs non-GvHD. Proteins that were present in >50% of at least one cohort and either differed (p<0.1) or were only present in one cohort were analyzed for network interaction and associated processes. Created with BioRender.com.

FIG. 5 depicts complement activation in GvHD with BOS vs no GvHD in samples collected ˜68 days before clinical diagnosis of BOS compared to controls without GvHD. MS Label free quantitation was used to quantitate plasma protein isoform expression. Pathway analysis was performed using Clarivate's MetaCore-GeneGo software analysis by inserting proteins that differed (p<0.05) between GvHD-BOS and non-GvHD. Schematic shows up-regulation of 9 components of complement pathways in GvHD with BOS compared to non-GvHD (cohort B vs A), in addition to up-regulation of complement inhibitors: Factor I, Vitronectin, and C1 inhibitor. Retrieved from https://app.biorender.com/biorender-templates.

FIG. 6 is a schematic showing HIF1a signaling pathway comparing (A) GvHD with BOS vs non-GvHD, (B) GvHD with BOS vs GvHD, and (C) GvHD vs non-GvHD at the time of BOS diagnosis. Non-biased proteomic analysis revealed differences (P<0.05) or the presences in one cohort and absence in the other of activators of HIF1α and inhibitors of its degradation are elevated at the time of BOS diagnosis, with highest levels in GvHD-BOS, indicating an upregulation of a protective pathway. Created with BioRender.com.

FIG. 7 shows ELISA measurement of CASPA8P2 in children (n=134) after HSCT. (A) Levels of CASPA8P2 (mean +95% CI) were undetectable 14 days after HSCT, but increased steadily through day 100, a conventional post-transplant observational timepoint. (B) Elevated CASPA8P2 was associated with increased risk of GvHD as in the proteomic analysis. GvHD, graft versus host disease, BOS, bronchiolitis obliterans syndrome; OS, overall survival.

FIG. 8 depicts ELISA validation of plasma protein biomarkers in banked samples 30 (A) and 100 (B) days post-HSCT. Odds ratios (OR) and corresponding 95% confidence intervals for bronchiolitis obliterans syndrome (BOS) and graft versus host disease (GvHD) grades 2-4 are shown. ILK, integrin-linked protein kinase; CASP8AP2, caspase 8 associated protein 2; SAC3D1, SAC3 domain-containing protein 1; IFIT1, Interferon Induced Protein with Tetratricopeptide Repeats 1; ATPase13, manganese transporting protein ATPase13A1. N=103-133, depending on timepoint.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the diagnosis and treatment of HSCT patients at risk of developing BOS or GvHD.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

Disclosed are methods for the personalized diagnosis of bronchiolitis obliterans syndrome and graft-versus-host-disease and therapies for their prevention or treatment in stem-cell transplant recipients, particularly in pediatric patients.

In one aspect, a method for identifying an individual at risk for developing bronchiolitis obliterans syndrome (BOS) after hematopoietic stem cell transplant (HSCT), is disclosed. The method may comprise
a) detecting one or more biomarker (or isoform thereof) selected from: integrin-linked protein kinase (ILK), Kelch-like protein 5 (KLHLS), kinesin-like protein 22 (KID), SAC3 domain-containing protein 1 (SAC3D1), RRP12-like protein (PRP12), manganese transporting protein, ATPase13A1 (ATP13A1), sorting nexin 8 (SNX8), and caspase 8 associated protein 2 (CASP8AP2, or FLASH), Interferon Induced Protein with Tetratricopeptide Repeats 1 (IFIT1), MTUS2 (microtubule associated tumor suppressor candidate 2), Factor I, Vitronectin, C1 inhibitor, Plasma protease C1 inhibitor precursor, complement component 1q (C1q), complement component 1s (C1s) (e.g., subcomponent isoform 1 preproprotein), complement component 1r (C1r), complement component 2 (C2) (e.g. isoform 5), complement component 3 (C3), complement component 4 (C4), complement component 4a (C4a), complement component 5 (C5) (e.g. isoform 2), complement component 7 (C7), complement component 8 (C8), and complement component 9 (C9),, Complement C3 preproprotein, Complement C4A (Rodger's blood group)-like preproprotein, Complement C4-B preproprotein,, Complement component C7 precursor, Complement component C8 alpha chain preproprotein, Complement component C9 preproprotein, Complement factor B preproprotein, Complement factor I (e.g., isoform X5), Complement receptor type 2 (e.g. isoform 1 precursor), Complement receptor type 1 (e.g. isoform X3), albumin, leukocyte elastase, mucin 5AC, Matrix metalloproteinase-9 (MMP-9), Mucin 5AC (Mucin 5 subtype AC, or MUC5AC), Matrix metalloproteinase-2 (MMP-2), leukocyte elastase, alpha-defensin, CAMP, histone H2, histone H2a, histone H3, histone H4, leukocyte elastase, PERM (PPARGC1 And ESRR Induced Regulator, Muscle 1), Factor B, and combinations thereof;
b) quantifying a level of one or more biomarkers detected in (a); and
c) comparing the level of said the or biomarkers to a control value;
   wherein a deviation in a level of said one or more biomarkers from said control value indicates said individual at risk for developing BOS.

The biomarker may be a protein biomarker, or, in other aspects, may be the corresponding gene of a protein biomarker that is detected via expression of a gene that encodes for the biomarker to be detected. In one aspect, the detecting of the biomarker may be carried out via enzyme-linked immunosorbent Assay (ELISA), western blotting, mass spectrometry, reverse transcription polymerase chain reaction (RT-PCR), northern blotting, in situ hybridization, microarrays, RNA sequencing (RNA-seq), Massively Parallel Signature Sequencing (MPSS), protein microarrays, or via detection of gene transcripts, such methods being known in the art. The biomarker may be detected in a biological sample obtained from the individual. In one aspect, the biological sample may be a bronchoalveolar lavage fluid (BALF) sample. In one aspect, the biological sample may be a blood, serum, or plasma sample from said individual.

In one aspect, the control value may be a level of said biomarker in said individual prior to HSCT. In one aspect, the control value may be a baseline value of the individual for the one or more biomarkers. The baseline value may be determined in the individual undergoing HSCT at a time point selected from prior to HSCT (e.g., within 10 years prior to HSCT, within 5 years prior to HSCT, within 2 years prior to transplant, within one year prior to HSCT, within six months prior to HSCT, within one month prior to HSCT, within two weeks prior to HSCT, within a week prior to HSCT, the day of or the day before transplant), or immediately following HSCT (i.e., within one day of HSCT, within two days of HSCT, within three days of HSCT, within four days of transplant, within five days of transplant, within six days of HSCT, within seven days of HSCT, within eight days of HSCT, within nine days of HSCT, within 10 days of HSCT. The baseline value may be an average of the levels of a biomarker obtained at various timepoints prior to HSCT and/or immediately following HSCT. Alternatively, the control value may be a value attributed to the average of a healthy population that is representative for that patient, i.e., a healthy, similarly aged individual of the same sex.

In one aspect, the detecting of step (a), which is carried out after HSCT for the purpose of determining a change in a biomarker level, may be carried out at a time point selected from day 14 post-HSCT, day 30 post-HSCT, day 60 post-HSCT, and day 100 post-HSCT.

In one aspect, where an increase in a biomarker is detected, as compared to a control value, such increase is indicative of the individual having a higher likelihood of developing BOS. In one aspect, where a decrease in a biomarker is detected, as compared to a control value, such decrease is indicative of the individual having a higher likelihood of developing BOS. Net, a deviation from a baseline or control value is indicative of the individual having a higher likelihood of developing BOS. In one aspect, when ATPase13A1 is decreased as compared to a control value, the individual is diagnosed as likely to develop BOS and is treated for BOS. In one aspect, when IFIT1 is increased as compared to a control value, the individual is diagnosed as likely to develop BOS and is treated for BOS.

The method of any preceding claim, further determining whether said individual has one or both of hypertension and thrombosis, wherein a diagnosis of one or both of hypertension and thrombosis is indicative of said individual being likely to develop BOS.

The method of any preceding claim, further determining whether said individual has dysregulation in one or more of vitamin transport, protein transport, angiotensin processing, thrombin regulation, platelet degranulation, acute inflammatory response, ERK1 and ERK2 signaling, proteolysis regulation, exocytosis regulation, iron transport regulation, wherein a determination of dysregulation is indicative of said individual being likely to develop BOS.

In one aspect, the individual may be diagnosed with graft versus host disease (GvHD). The individual may be a pediatric individual or an adult.

In one aspect, where the individual is diagnosed as likely to develop BOS, one or more agents may be administered to the individual, the one more agents being selected from beta blockers, antihyperlipidemic-HMG CoA reductase inhibitors (statins), macrolide antibiotics, anthracycline conjugates, small molecule elastase inhibitors, angiogenesis inhibitors, tetracycline antibiotics, mucolytics, matrix metalloprotease inhibitors, and combinations thereof.

In one aspect, the agent is a beta blocker and is selected from sotalol (Betapace), metoprolol (Lopressor, Toprol-XL), atenolol (Tenormin), propranolol (Inderal), nadolol (Corgard), timolol (Blocadren), pindolol (Visken), carvedilol (Coreg), labetalol (Trandate), penbutolol (Levatol), bisoprolol (Zebeta),, esmolol (Brevibloc), acebutolol (Sectral), betaxolol (Kerlone), carvedilol (Coreg), labetalol (Trandate).

In one aspect, the agent is an antihyperlipidemic-HMG CoA reductase inhibitor (statin) and is selected from pravastatin (Pravachol), atorvastatin (Lipitor), fluvastatin (Lescol), lovastatin (Mevacor, Altoprev), rosuvastatin (Crestor), simvastatin (Zocor), pitavastatin (Livalo), and combinations thereof.

In one aspect, the agent is a macrolide antibiotic and is selected from Azithromycin (Zithromax, Zmax), Clarithromycin (Biaxin), Erythromycin (Ery-Tab, Erythrocin), Fidaxomicin (Dificid), Josamycin (Josacine), Roxithromycin (Surlid, Rulid), Spiramycin (Rovamycin), and combinations thereof.

In one aspect, the agent is an anthracycline conjugate and is selected from aldoxorubicin, doxorubicin hydrochloride, SGN-CD33A (vadastuximab talirine), DMA-Doxorubicin (ADCT-602), gemtuzumab ozogamicin (Mylotarg), and combinations thereof.

In one aspect, the agent is an elastase inhibitor and is selected from alvelestat, freselestat, ZD8321, elafin, secretory leukocyte protease inhibitor (SLPI), alpha-1 antitrypsin (AAT), SerpinA3, SerpinB1, sivelestat, AZD9668, ONO-6818, BAY-849, tosedostat, L-658,758, FICZ, PF-06741086, AZD7986, RO5461111, SPK-3009, KBP-7072, ONO-5046, GW311616A, GW746027, and combinations thereof.

In one aspect, the agent is an angiogenesis inhibitor and is selected from batimastat, bevacizumab (Avastin), aflibercept (Zaltrap), ramucirumab (Cyramza), ziv-aflibercept (Zaltrap), sunitinib (Sutent), sorafenib (Nexavar), pazopanib (Votrient), regorafenib (Stivarga), cabozantinib (Cabometyx, Cometriq), everolimus (Afinitor, Zortress), cilengitide (EMD 121974), thalidomide (Thalomid), lenalidomide (Revlimid), pomalidomide (Pomalyst), TNP-470, endostatin, suramin, and combinations thereof.

In one aspect, the agent is a tetracycline antibiotic and is selected from doxycycline, minocycline, tetracycline, demeclocycline, tigecycline, oxytetracycline, methacycline, rolitetracycline, and combinations thereof.

In one aspect, the agent is a mucolytic and is selected from recombinant human deoxyribonuclease (pulmozyme), acetylcysteine, ambroxol, bromhexine, carbocisteine, erdosteine, hypertonic saline, and combinations thereof.

In one aspect, the agent is a matrix metalloprotease inhibitor and is selected from marimastat, ONO4817, rebimastat, tanomastat, S-3304, and combinations thereof.

In one aspect, a plurality of detection agents specific for two or more biomarkers are disclosed herein. Such detections agents may include antibodies capable of detecting one or more antibodies, or oligonucleotides specific for RNA that encodes for one or more biomarker. Further provided are kits for carrying out the methods, which may include one or more of an ELISA plate pre-coated with antibodies specific for one or more biomarkers, sample collection containers and reagents for sample preparation or preservation, RNA extraction reagents, PCR reagents including primers specific for one or more biomarkers, and a control reagent (positive and negative control reagent).

In one aspect, a method of treating an individual having or likely to develop BOS, is disclosed, the method comprising administering an active agent selected from a beta blocker, an antihyperlipidemic-HMG CoA reductase inhibitor (statin), an angiogenesis inhibitor, a mucolytic, a matrix metalloprotease inhibitor, an anthracycline conjugate, a tetracycline antibiotic, a small molecule elastase inhibitor, a macrolide antibiotic, and combinations thereof. The administration may be carried out for a period of time and at a dosage sufficient to achieve the desired result, i.e., prevention of, delayed progression of, or resolution of BOS in an individual having undergone HSCT.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

Patients with a clinical diagnosis of BOS and available plasma samples were identified in an institutional HSCT repository. 21 patients representing 3 cohorts were studied: 7 patients with GvHD-BOS (Cohort B, designated cases); 7 sex, age ±2 years of age, and timepoint matched patients with severe grade 3-4 GvHD without BOS (Cohort C); and 7 sex, age ±2 years and timepoint matched transplant recipient controls with no GvHD or BOS (Cohort A). Cohorts A and C were both considered controls. GvHD diagnosis was made by the treating physician, and the diagnosis was supported with tissue biopsies whenever feasible. The entire proteomics analyses included 42 samples, with each participant providing a sample at two timepoints: one timepoint was designated “prior to diagnosis” and was a median of 68 (38-188) days before diagnosis of BOS in cases with BOS. Samples analyzed from controls for the “prior to diagnosis” timepoint were the available sample closest to the timepoint after transplant of the “prior to diagnosis” sample in their matched case. The second timepoint, termed “time of diagnosis” (median 203 days after transplant; range 92-361) was a sample collected close to the time of diagnosis in cases, and the closest sample available at a similar post-transplant timepoint for the matched controls.

Proteomic Discovery. Initial discovery MS analyses of a total of 14,107 HSCT plasma protein isoforms found 12,629 (no GvHD vs. GvHD-BOS, cohorts A vs. B), 12,726 (no GvHD vs. GvHD, cohorts A vs. C), or 12,275 (GvHD-BOS vs GvHD, cohorts B vs. C) isoforms were present in at least 50% of compared cohorts at time of diagnosis. Prior to diagnosis, analyses of a total of 10,963 HSCT plasma protein isoforms found 9,784 (A vs. B), 9,748 (A vs. C), or 8,639 (B vs. C) isoforms were present in at least 50% of compared cohorts. At time of diagnosis, considering proteins that were present in at least 50% of compared cohorts, the following subsets of isoforms were differentially expressed (P<0.05): 1,734 in cohorts A vs. C (no GvHD vs. GvHD); 1,199 isoforms in cohorts A vs. B (no GvHD vs. GvHD-BOS); and 311 isoforms in cohorts C vs. B (GvHD vs. GvHD-BOS). Notably, 684 isoforms are detected only in GvHD, and 602 only in GvHD-BOS vs. no GvHD control. Prior to diagnosis, considering proteins that were present in at least 50% of compared cohorts, the following subsets of isoforms were differentially expressed (P<0.05): 1,263 in cohorts A vs. C (no GvHD vs. GvHD); 1,439 isoforms in cohorts A vs. B (no GvHD vs GvHD-BOS); and 359 isoforms in cohorts C vs. B (GvHD vs. GvHD-BOS). 509 isoforms are detected only in GvHD, and 499 only in GvHD-BOS vs. no GvHD control.

### Network Analysis: Prior to Diagnosis Timepoint

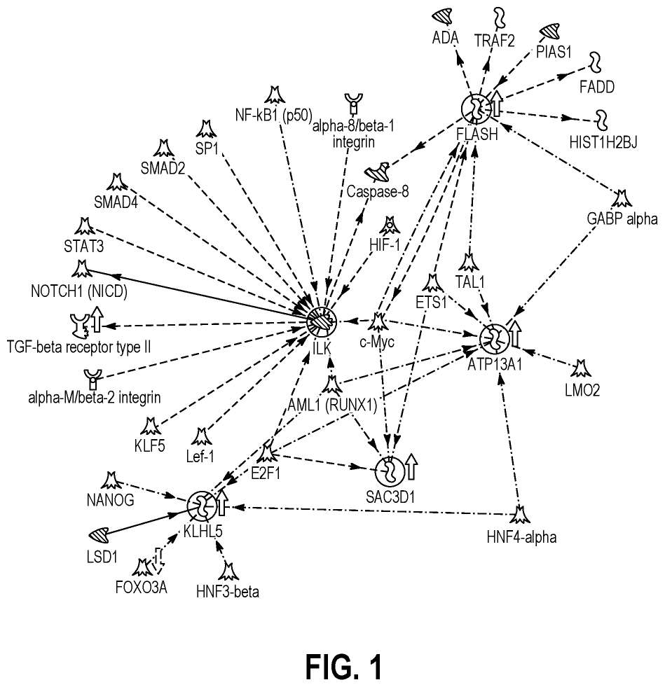

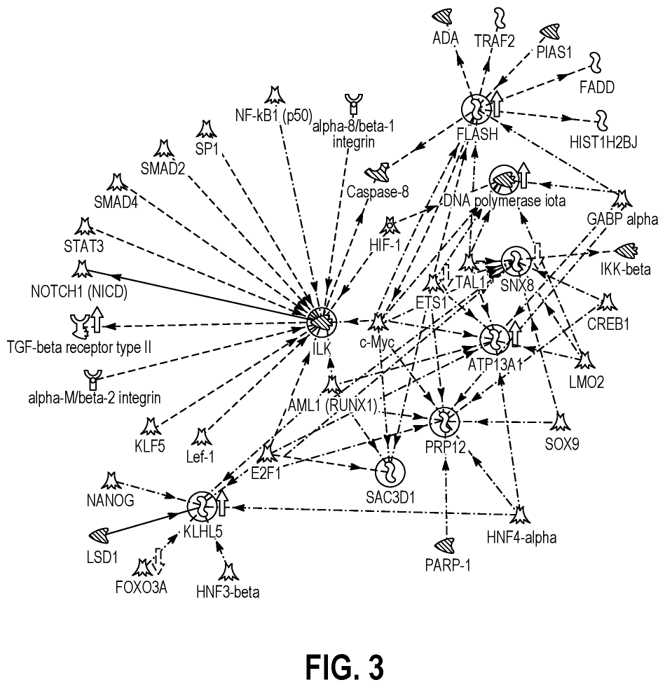

A number of markers clustered around several networks that predict features of GvHD without BOS and/or GvHD-BOS. In GvHD without BOS compared with no GVHD (cohort C vs A), changes in plasma levels of integrin-linked protein kinase (ILK), Kelch-like protein 5 (KLHL5), kinesin-like protein 22 (KID), SAC3 domain-containing protein 1 (SAC3D1), RRP12-like protein (PRP12), manganese transporting protein ATP-ase13A1 (ATP13A1), sorting nexin 8 (SNX8), and caspase 8 associated protein 2 (CASP8AP or FLASH) (FIGS. 1-3) indicated alterations in networks of hypoxia (p=1.98×10⁻²³, FDR=1.68×10⁻²⁰) and respiratory disease (p=5.82×10⁻²², FDR=2.48×10⁻¹⁹).

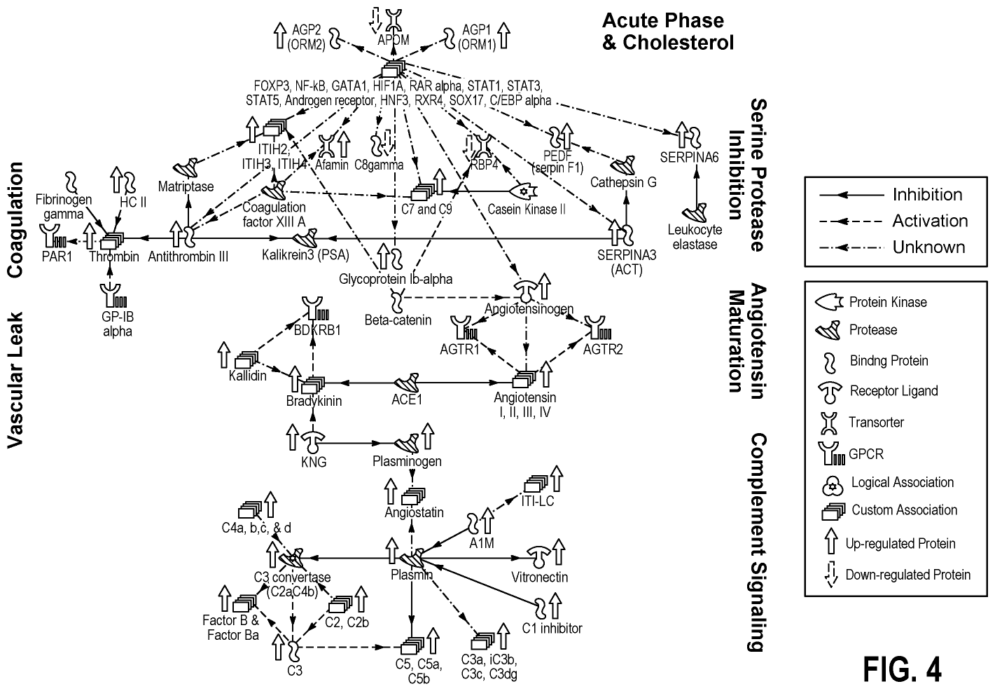

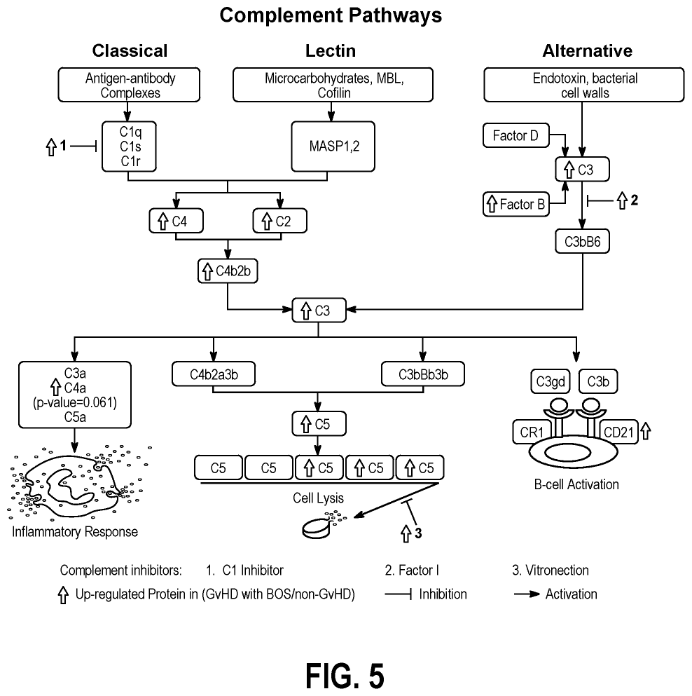

MS proteomics detected differences in proteins that highly enriched process networks in both GvHD-BOS and GvHD vs. no GvHD (cohorts B and C vs. cohort A) including complement activation (p=7.75×10⁻³⁹, FDR=3.79×10⁻³⁷), kallikrein-kinin signaling (p=4.47×10⁻¹⁷, FDR=1.09×10⁻¹⁵), blood coagulation (p=9.64×10⁻¹¹, FDR=1.57×10⁻⁹), and platelet-endothelium-leucocyte interactions (1.12×10⁻⁶, FDR=1.37×10⁻⁵). Our data suggest that proteins that segregate BOS from no GvHD cluster into coagulopathy, serine protease inhibition, vascular leak, angiotensin maturation and complement signaling, highlighting important clinical features of BOS such as inflammation and tissue injury (FIG. 4).

### Differential Complement Signaling

Nine complement factors are significantly elevated in prior to diagnosis samples (Table 2), identifying important changes in complement pathway signaling (FIG. 4) in both GvHD-BOS and GvHD vs. no GvHD. Complement activation alone did not discriminate GvHD from GvHD-BOS prior to (not shown) or at time of diagnosis (Table 3). However, complement C2 isoform 5 and C4-A proteins and the kinase ILK were present in patients that would go on to develop GvHD-BOS but not in those that would present with GvHD (Table 4). Furthermore, inhibitors of complement signaling, such as C1 inhibitor, factor I, and vitronectin were significantly elevated, suggesting feedback mechanisms are activated to control what is likely chronic stimulation of complement signaling in both GvHD and GvHD-BOS (Table 4).

TABLE 2
Alterations of complement system proteins
for the prior to diagnosis samples

| | (GvHD-BOS | | (GvHD vs | |
|---|---|---|---|---|
| | vs no GvHD) | | no GvHD) | |
| | fold | p- | fold | p- |
| Protein Isoform | change | value | change | value |
| Complement C2 isoform 5 | 2.53 | 0.001 | 1.87 | 0.135 |
| Complement C1s | 1.43 | 0.202 | 2.12 | 0.020 |
| subcomponent isoform 1 | | | | |
| preproprotein | | | | |
| Complement C3 preproprotein | 1.63 | 0.004 | 1.63 | 0.043 |
| Complement C4A (Rodger's | 2.29 | 0.061 | 1.75 | 0.118 |
| blood group)-like | | | | |
| preproprotein | | | | |
| Complement C4-B | 2.57 | 0.018 | 2.61 | 0.004 |
| preproprotein | | | | |
| Complement C5 isoform 2 | 2.07 | 0.021 | 2.77 | 0.022 |
| Complement component C7 | 1.62 | 0.010 | 2.08 | 0.062 |
| precursor | | | | |
| Complement component C8 | 1.76 | 0.028 | 2.05 | 0.026 |
| alpha chain preproprotein | | | | |
| Complement component C9 | 2.03 | 0.011 | 2.51 | 0.014 |
| preproprotein | | | | |
| Complement factor B | 2.52 | 0.003 | 2.32 | 0.009 |
| preproprotein | | | | |
| Complement factor I isoform | 1.74 | 0.046 | 1.85 | 0.059 |
| X5 | | | | |
| Complement receptor type 2 | 1.61 | 0.016 | 1.77 | 0.003 |
| isoform 1 precursor | | | | |
| Complement receptor type 1 | 1.12 | 0.265 | 1.17 | 0.038 |
| isoform X3 | | | | |

TABLE 3
Alterations of complement system proteins at time of diagnosis.

| | (GvHD-BOS/ | | (GvHD/ | | (GvHD-BOS/ | |
|---|---|---|---|---|---|---|
| Protein | no GvHD) | p- | no GvHD) | p- | GvHD) | p- |
| Isoform | fold change | value | fold change | value | fold change | value |
| Complement C2 | 1.33 | 0.019 | N/A* | N/A* | N/A* | N/A* |
| isoform 5 | | | | | | |
| Complement C4-A | 1.43 | 0.110 | 3.06 | 0.019 | 0.47 | 0.035 |
| isoform 2 | | | | | | |
| preproprotein | | | | | | |
| Complement | 1.22 | 0.014 | 0.96 | 0.62 | 1.28 | 0.024 |
| factor I | | | | | | |
| isoform X4 | | | | | | |

*indicates that protein was not detected in GvHD group.

TABLE 4
MS proteomic measurement of plasma protein markers of GvHD and GvHD with BOS.

| | (GvHD-BOS/ | | (GvHD/ | | (GvHD-BOS/ | | (GvHD/ | |
|---|---|---|---|---|---|---|---|---|
| Protein | no GvHD) | p- | no GvHD) | p- | no GvHD) | p- | no GvHD) | p- |
| Isoform | fold change | value | fold change | value | fold change | value | fold change | value |
| Vitronectin | 2.46 | 2.89E−04 | 2.08 | 0.010 | 1.93 | 0.017 | 1.66 | 0.173 |
| precursor | | | | | | | | |
| Plasma | 2.52 | 1.04E−05 | 2.88 | 0.022 | 2.50 | 0.012 | 2.98 | 4.38E−07 |
| protease | | | | | | | | |
| C1 | | | | | | | | |
| inhibitor | | | | | | | | |
| precursor | | | | | | | | |
| CASP8- | 1.15 | 0.077 | 1.37 | 0.006 | 1.17 | 0.107 | 1.17 | 0.080 |
| associated | | | | | | | | |
| protein 2 | | | | | | | | |

### Differential HIF1α (Hypoxia-Inducible Factor 1 Alpha) Signaling

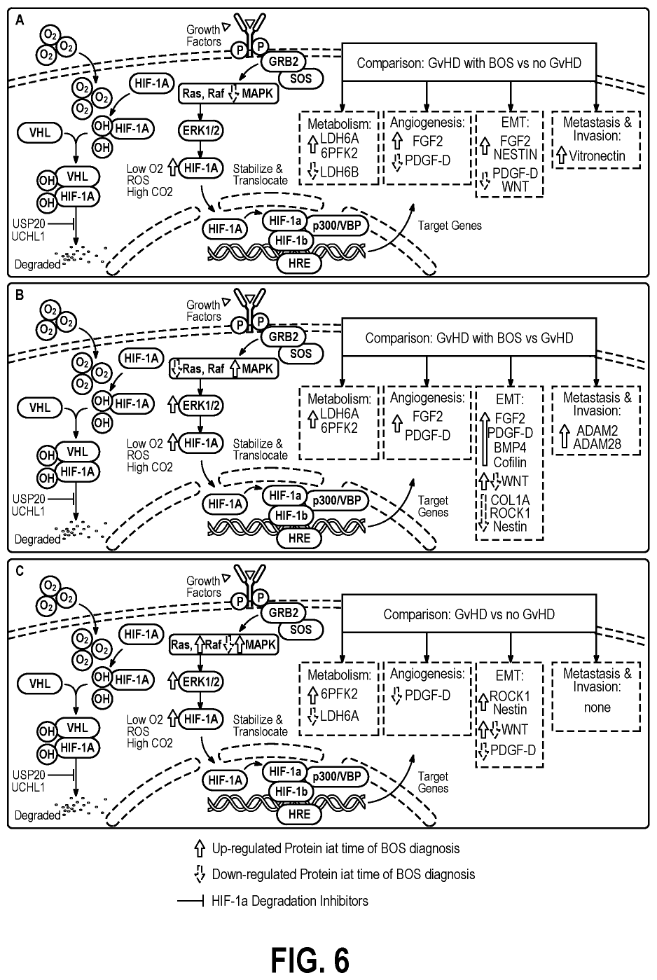

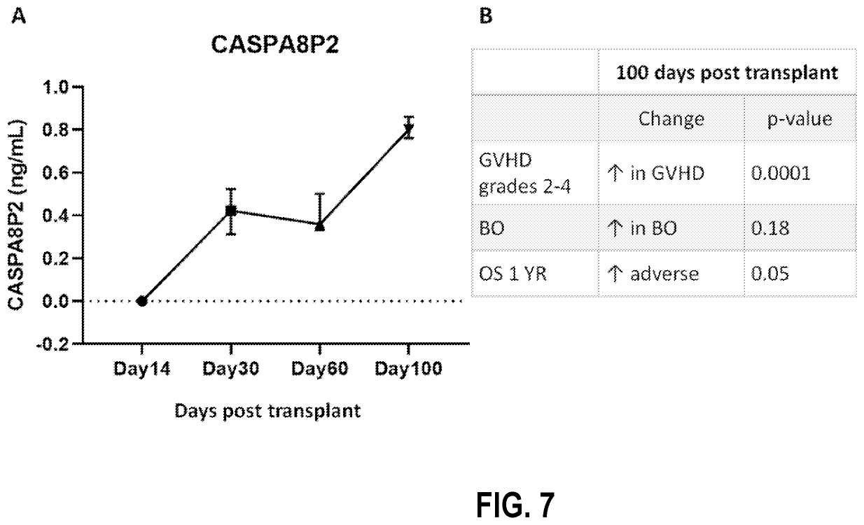

HIF1α activation was enriched in GvHD-BOS compared with no GvHD or GvHD alone. Samples banked from patients at the time of diagnosis timepoint showed elevation in the levels of the HIF1a activators MAPK1/ERK1 and MAPK2/ERK2 and HIF1a degradation inhibitor ubiquitin carboxyl-terminal hydrolase isozyme L1 (FIG. 6). HIF1α functions as a master transcriptional regulator of the adaptive and protective response to hypoxia, and its activation in patients that go on to present with BOS may indicate upregulation of the protective pathway in response to early airway obstruction and hypoxemia.

### ELISA Validation of Markers Discovered by MS

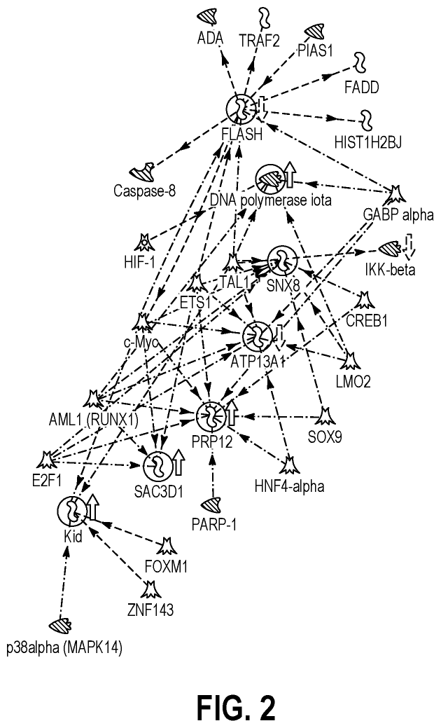

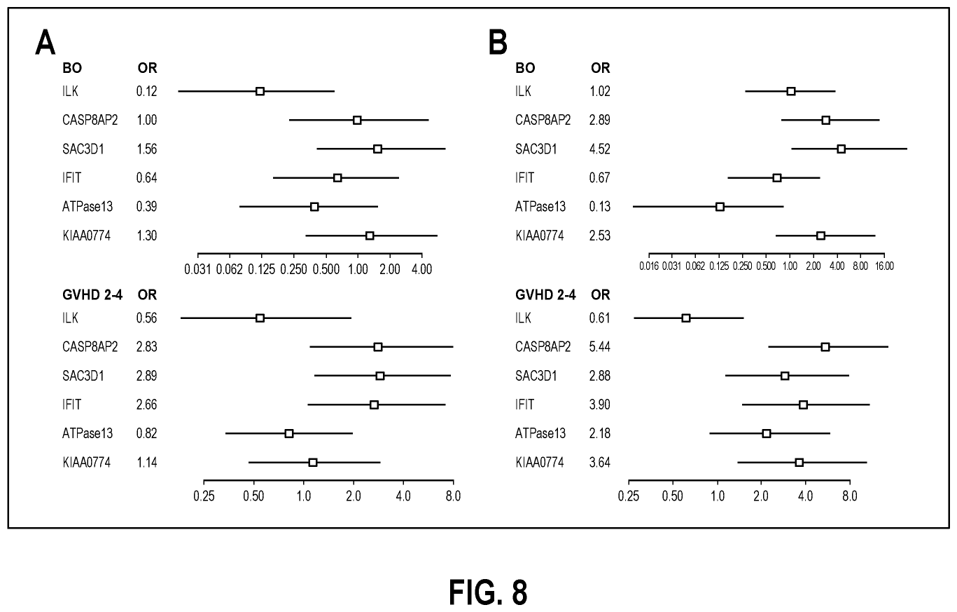

Cross-platform validation of the protein caspase 8 associated protein 2 (FLASH in FIG. 2) in a cohort of >100 consecutive HSCT patients is shown in FIG. 6. Levels of CASPSAP2 were measured by ELISA at days 14 (n=107), 30 (n=108), 60 (n=108), and 100 (n=134) after HSCT. Levels were undetectable at 14 days after HSCT but increased steadily through day 100 (FIG. 6A). Day 100 levels were significantly elevated in those with GvHD, and were similarly elevated in those with BOS, but lacked statistical significance, likely due to smaller number of BOS cases (FIG. 6B). ELISA data confirmed that ILK, CASP8AP2, SAC3D1, IFIT1, ATPase13, and KIAAA0774 ILK (Integrin Linked Kinase), CASP8AP2 (Caspase 8 Associated Protein 2), SAC3D1 (Sac3 Domain-Containing Protein 1), IFIT1 (Interferon-Induced Protein with Tetratricopeptide Repeats 1), ATPase13 (ATPase 13A1), KIAAA0774 (no full name available; non-standard gene name) plasma levels correlated with GvHD and/or GvHD-BOS, in some cases as early as 14 days post-HSCT (FIG. 8).

### Drug Interactions with Altered Pathways

The proteomic data provided a detailed picture of the pathways implicated in the phenotypes of each examined cohort, indicating that statin and azithromycin therapies (Table 5) would be beneficial in reducing the lung disease exhibited by subjects that develop BOS. Furthermore, we discovered an association of BOS with increased IFN-γ signaling and expression of fibronectin binding integrins in samples collected prior to the diagnosis of BOS, identifying additional potential therapeutic targets.

TABLE 5
MS Therapeutic Drug Analysis Results

| Drug (Site of | | | Gene |
|---|---|---|---|
| Action) | Effect | Target Protein Name | Symbol |
| Solatol | Inhibition | Potassium voltage-gated channel | KCNH2 |
| (extracellular) | | subfamily H member 2 | |
| Atorvastatin | Inhibition | 3-hydroxy-3-methylgltaryl- | HMGCR |
| (intracellular) | | coenzyme A reductase | |
| Axithromycin | Inhibition | Mucin-5AC | MUC5AC |
| (intracellular) | | | |
| Pravastatin | Inhibition | 3-hydroxy-3-methylgltaryl- | HMGCR |
| (intracellular) | | coenzyme A reductase | |

ELISA experiments validated isoforms identified by proteomics as altered in GvHD and in some instances BOS, for example CASP8AP, supporting the discovery process. Importantly, ATPase13A1 was reduced in children with GvHD who later developed BOS but was unchanged in those with GvHD and who did not develop BOS. IFIT1 was elevated in those with GvHD but no BOS, and unchanged in those with GvHD who later developed BOS

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method for identifying an individual at risk for developing bronchiolitis obliterans syndrome (BOS) after hematopoietic stem cell transplant (HSCT), comprising
a. detecting one or more biomarker (or isoform thereof) selected from: integrin-linked protein kinase (ILK), Kelch-like protein 5 (KLHLS), kinesin-like protein 22 (KID), SAC3 domain-containing protein 1 (SAC3D1), RRP12-like protein (PRP12), manganese transporting protein, ATPase13A1 (ATP13A1), sorting nexin 8 (SNX8), and caspase 8 associated protein 2 (CASP8AP2, or FLASH), Interferon Induced Protein with Tetratricopeptide Repeats 1 (IFIT1), MTUS2 (microtubule associated tumor suppressor candidate 2), Factor I, Vitronectin, C1 inhibitor, Plasma protease C1 inhibitor precursor, complement component 1q (C1q), complement component 1s (C1s), complement component 1r (C1r), complement component 2 (C2), complement component 3 (C3), complement component 4 (C4), complement component 4a (C4a), complement component 5(C5), complement component 7 (C7), complement component 8 (C8), and complement component 9 (C9), Complement C3 preproprotein, Complement C4A (Rodger's blood group)-like preproprotein, Complement C4-B preproprotein, Complement component C7 precursor, Complement component C8 alpha chain preproprotein, Complement component C9 preproprotein, Complement factor B preproprotein, Complement factor I, Complement receptor type 2, Complement receptor type 1, albumin, leukocyte elastase, mucin 5AC, Matrix metalloproteinase-9 (MMP-9), Mucin 5AC (Mucin 5 subtype AC, or MUC5AC), Matrix metalloproteinase-2 (MMP-2), leukocyte elastase, alpha-defensin, CAMP, histone H2, histone H2a, histone H3, histone H4, leukocyte elastase, PERM (PPARGC1 And ESRR Induced Regulator, Muscle 1), Factor B, and combinations thereof;
b. quantifying a level of said one or more biomarkers detected in (a); and
c. comparing said level of said one or biomarkers to a control value;
wherein a deviation in a level of said one or more biomarkers from said control value indicates said individual at risk for developing BOS.

2. The method of claim 1 wherein said control value is a level of said biomarker in said individual prior to HSCT.

3. The method of claim 1 wherein said control value is a level of said biomarker in said individual prior to HSCT, within a day of HSCT, within two days of HSCT, within three days of HSCT, within four days of HSCT, within five days of HSCT, within six days of HSCT, within seven days of HSCT, within eight days of HSCT, within nine days of HSCT, within 10 days of HSCT.

4. The method of claim 1 wherein said detecting of step (a) is carried out at a time point selected from day 14 post-transplant, day 30 post-transplant, day 60 post-transplant, and day 100 post-transplant.

5. The method of claim 1, wherein an increase in said biomarker as compared to a control value is indicative of said individual having a higher likelihood of developing BOS.

6. The method of claim 1, wherein a decrease in said biomarker as compared to a control value is indicative of said individual having a higher likelihood of developing BOS.

7. The method of claim 1, wherein where ATPase13A1 is decreased as compared to said control value, said individual is diagnosed as likely to develop BOS and is treated for BOS.

8. The method of claim 1, wherein where IFIT1 is increased as compared to said control value, said individual is diagnosed as likely to develop BOS and is treated for BOS.

9. The method of claim 1, wherein said biomarker is detected in a biological sample obtained from said individual.

10. The method of claim 9, wherein said sample is selected from a plasma sample, serum sample, blood sample, bronchoalveolar lavage fluid (BALF) sample, and combinations thereof.

11. The method of claim 9, wherein said sample is obtained at one or more time points selected from prior to transplant, day 14 post-transplant, day 30 post-transplant, day 60 post-transplant, and day 100 post-transplant.

12. The method of claim 1, further determining whether said individual has one or both of hypertension and thrombosis, wherein a diagnosis of one or both of hypertension and thrombosis is indicative of said individual being likely to develop BOS.

13. The method of claim 1, further determining whether said individual has dysregulation in one or more of vitamin transport, protein transport, angiotensin processing, thrombin regulation, platelet degranulation, acute inflammatory response, ERK1 and ERK2 signaling, proteolysis regulation, exocytosis regulation, iron transport regulation, wherein a determination of dysregulation is indicative of said individual being likely to develop BOS.

14. The method of claim 1, wherein said individual is diagnosed with graft versus host disease (GvHD).

15. (canceled)

16. (canceled)

17. The method of claim 1, wherein said individual is diagnosed as likely to develop BOS, and is administered an agent selected from a beta blocker, and antihyperlipidemic-HMG CoA reductase inhibitor (statin), a macrolide antibiotic, an anthracycline conjugate, a small molecule elastase inhibitor, an angiogenesis inhibitor, a tetracycline antibiotic, a mucolytic, a matrix metalloprotease inhibitor, and combinations thereof.

18. The method of claim 17 wherein said agent is a beta blocker and is selected from sotalol (Betapace), metoprolol (Lopressor, Toprol-XL), atenolol (Tenormin), propranolol (Inderal), nadolol (Corgard), timolol (Blocadren), pindolol (Visken), carvedilol (Coreg), labetalol (Trandate), penbutolol (Levatol), bisoprolol (Zebeta), esmolol (Brevibloc), acebutolol (Sectral), betaxolol (Kerlone), carvedilol (Coreg), labetalol (Trandate).

19. The method of claim 17 wherein said agent is an antihyperlipidemic-HMG CoA reductase inhibitor (statin) and is selected from pravastatin (Pravachol), atorvastatin (Lipitor), fluvastatin (Lescol), lovastatin (Mevacor, Altoprev), rosuvastatin (Crestor), simvastatin (Zocor), pitavastatin (Livalo), and combinations thereof.

20. The method of claim 17 wherein said agent is a macrolide antibiotic and is selected from Azithromycin (Zithromax, Zmax), Clarithromycin (Biaxin), Erythromycin (Ery-Tab, Erythrocin), Fidaxomicin (Dificid), Josamycin (Josacine), Roxithromycin (Surlid, Rulid), Spiramycin (Rovamycin), and combinations thereof.

21. (canceled)

22. (canceled)

23. (canceled)

24. (canceled)

25. (canceled)

26. (canceled)

27. A diagnostic test comprising a plurality of detection agents, said plurality of detection agents specific for one or more biomarker of claim 1.

28. A method of treating an individual having or likely to develop BOS, comprising administering an active agent selected from a beta blocker, an antihyperlipidemic-HMG CoA reductase inhibitor (statin), an angiogenesis inhibitor, a mucolytic, a matrix metalloprotease inhibitor, an anthracycline conjugate, a tetracycline antibiotic, a small molecule elastase inhibitor, a macrolide antibiotic, and combinations thereof.
